(19) United States
(12) Patent Application Publication
(10) Pub. No.: US 2011/0145951 A1
(43) Pub. Date: Jun. 16, 2011

(54) TRANSGENIC SWEET SORGHUM WITH ALTERED LIGNIN COMPOSITION AND PROCESS OF PREPARATION THEREOF

(75) Inventors: Asitava Basu, Kharagpur (IN); Mrinal K. Maiti, Kharagpur (IN); Satarupa Kar, Kharagpur (IN); Soumitra K. Sen, Kharagpur (IN); Banibrata Pandey, Hyderabad (IN)

(73) Assignee: NAGARJUNA ENERGY PRIVATE LIMITED, Hyderabad (IN)

(21) Appl. No.: 12/860,679

(22) Filed: Aug. 20, 2010

Related U.S. Application Data
(63) Continuation-in-part of application No. 12/545,699, filed on Aug. 21, 2009.
Application No. 12/545,699 is a continuation of application No. PCT/IB2008/000380, filed on Feb. 20, 2008.

(30) Foreign Application Priority Data
Feb. 21, 2007 (IN) 1481/CHE/2006

(51) Int. Cl. A01H 5/00; C12N 15/54; C12N 15/63; C07H 21/00

(52) U.S. Cl. 800/298; 536/23.2; 435/320.1; 536/24.33

(57) ABSTRACT

The present invention provides a sweet sorghum plant characterized by altered lignin content and/or altered lignin composition compared to a wild plant and this is achieved by manipulating the expression of caffeoyl-CoA-O-methyltransferase (CCoAOMT) in sweet sorghum by incorporation of a gene silencing construct comprising an isolated DNA sequence represented by SEQ ID NO 1.

### RELATED APPLICATION DATA

This application is a continuation-in-part of U.S. application Ser. No. 12/545,699 filed on Aug. 21, 2009, which is a continuation under 35 U.S.C. §120 of International Patent Application No. PCT/1132008/000380 filed on Feb. 20, 2008, which claims priority to Indian Patent Application No. 1481/CHE/2006 filed on Feb. 21, 2007. These disclosures are incorporated herein by reference in their entirety.

### FIELD OF THE INVENTION

The present invention is in the field of biotechnology and more particularly development of a plant having altered lignin composition.

### BACKGROUND AND PRIOR ART

Sweet sorghum, Sorghum bicolor (L.) Moench is the only crop that provides grain and stem that can be used for the production of alcohol, sugar, syrup, fuel etc. Sweet sorghum offers following advantages over the other crops:
Growing period (about 4 months) and water requirement (8000 m³ over two crops) are 4 times lower than those of sugarcane (12 to 16 months and 36000 m³ respectively).
Cost of cultivation of sweet sorghum is 3 times lower than sugarcane.
Seed propagated.
Suitable for mechanized crop production.
The ethanol production process from sweet sorghum is eco-friendly compared to that from molasses.
Ethanol burning quality is superior—less sulphur than from sugarcane and high octane rating.

See International Crops Research Institute for the Semi-Arid Tropics (ICRISAT) brochure, “Sweet Sorghum: Food, Feed, Fodder and Fuel Crop,” published 2006.

But the primary concern for using this plant is the presence of lignin, which adversely affects the process of extraction of beneficial materials. Beside the sugary juice, the stem offer great potential for biofuel production. However, due to the presence of lignin, as in other crop plants, affect the separation of the available sugars such as xylose, arabinose, glucose etc.

Lignin is a complex phenylpropanoid polymer. Plants comprise about 25-30% lignin based on the dry weight. Lignin is primarily deposited in the cell walls of supporting and conductive tissues, such as fibers and tracheary elements. The mechanical rigidity of lignin strengthens these tissues so that the tracheary elements can endure the negative pressure generated from transpiration without collapse of the tissue.

However, lignin is unfavorable in various other aspects. Lignin decreases the digestibility of animal forage crops and must be removed during pulping and paper making, which requires the use of chemicals hazardous to the environment. Lignin also appears to have a negative impact on the utilization of plant and tree biomass.

Lignin is considered to be dehydrogenatively polymerized from the monolignols p-coumaryl alcohol, coniferyl alcohol, and sinapyl alcohol. These monolignols are synthesized through the phenylpropanoid pathway. Structurally these monolignols differ only by the methoxy group at the 3C and 5C positions of the aromatic ring. Varying proportion of the monolignols determine the type of lignin such as H, G, S lignin. G lignin offers more resistance than S during enzymatic saccharification. G lignins are more condensed due to more numbers of intermolecular linkages, thereby showing more resistance.

Therefore, it is desirable to develop a sweet sorghum plant with altered lignin composition, said composition comprising more of S and less G lignin as compared to the wild. In order to achieve that it is also required that the modified plant must have more biomass content and its bio-chemical architecture is favorable for downstream processing.

Several approaches have been taken to decrease or alter the composition of lignin content to increase S/G ratio. However, the results have found to be contradictory, possibly due to lack of understanding of lignin biosynthetic pathway and due to inappropriate suitable approaches for down regulation of the lignin biosynthetic enzyme activity including choice of transgene, promoter used, construction of cassettes and above all, selection of transformants. Regulation of early steps enzymes like phenylalanine ammonia lyase, cinnamate 4-hydroxylase, 4-hydroxycinnamate CoA ligase reduced lignin content. However, it leads to pleiotropic effects including altered leaf shape, localized fluorescent lesion, stunted growth, reduced pollen activity, altered flower morphology and pigmentation, reduced level of soluble phenylpropanoids, decrease in S/G ratio etc (Elkind et al, 1990; Bate et al, 1994; Sewalt et al, 1997). Similar effects by other workers to alter or modify the S/G ratio have resulted in phenotypically defective plants. It was demonstrated that down regulation of caffeic acid O-methyltransferase activity could result dramatic decrease in syringyl lignin biosynthesis but with little effect on the synthesis of guaiacyl lignin, which is undesirable as the latter are more resistant to chemical degradation. However, till date any alteration in lignin composition in sweet sorghum is carried out primarily by down regulating COMT gene.

It is therefore of interest to develop a modified sweet sorghum plant having altered lignin composition and biochemically suitable for downstream processing. In order to achieve the same, the inventor had attempted to find out genes capable of altering lignin composition in sweet sorghum plant and said plant is susceptible to easy enzymatic degradation compared to control plant as well as COMT down-regulated sorghum plants generated in the present study through similar gene silencing approach.

### SUMMARY OF THE INVENTION

The present invention is directed towards a polypeptide sequence capable of altering the lignin composition in sweet sorghum. Down regulation of said polynucleotide in sweet sorghum plants causes formation of more S-lignin as compared to G-lignin, higher free sugar content and shows better morphological characteristics in modified plants as compared to the wild plant. Computer-based analysis of the isolated sequence reveals a similarity with CCoAOMT of arthologous plants. However, to date no literature discloses the presence of CCoAOMT and its role in modulation of lignin composition in sweet sorghum. Hereinafter the polynucleotide sequence is referred to as SEQ ID NO 1, which has CCoAOMT-like properties, whose sequence has been partially identified.

According to one aspect of the invention, an isolated polynucleotide sequence is provided, where the sequence is obtained from Sorghum bicolor encoding Caffeoyl-CoA-O-methyltransferase (CCoAOMT), and is represented by SEQ ID NO 1. According to some aspects, the DNA may be cDNA.

An additional aspect of the invention relates to a RNAi mediated gene silencing construct comprising a suitable fragment of nucleic acid represented by SEQ ID NO 1, where the nucleic acid is operably associated with a promoter, wherein the nucleic acid fragment is in the double stranded sense-antisense orientation and facilitate in vivo production of RNAi to at least a portion of a SEQ ID NO 1. Vectors comprising the construct are provided in a further aspect of the invention, and in some aspects of the invention the vector may be a pUC18 vector.

A further aspect of the invention relates to forward and reverse primers represented by SEQUENCE ID NO 3 and 4. The primers are useful in amplifying CCoAOMT.

Surprisingly, it has been found that down regulation of SEQ ID NO 1 in sweet sorghum results in a plant that is superior to the plants obtained by down regulating COMT in respect of the biomass, starch, and free sugar content and phenotypical properties.

### BRIEF DESCRIPTION OF FIGURES

FIG. 1: Enhanced growth of the anti-CCoAOMT-like transformed sorghum T0 plant (right) in comparison to control plant (left).

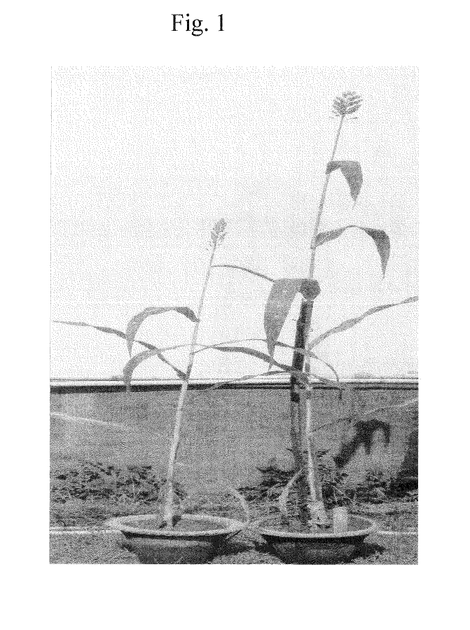

FIG. 2: Enhanced growth of transgenic sorghum (T1 plant) with down regulated CCoAOMT-like gene represented by SEQ ID NO 1 in field.

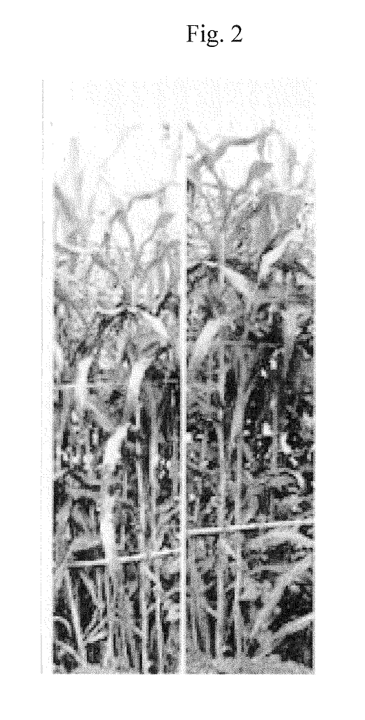

FIG. 3: Panicle of control plant (left) and CCoAOMT-like gene transformed plant (right).

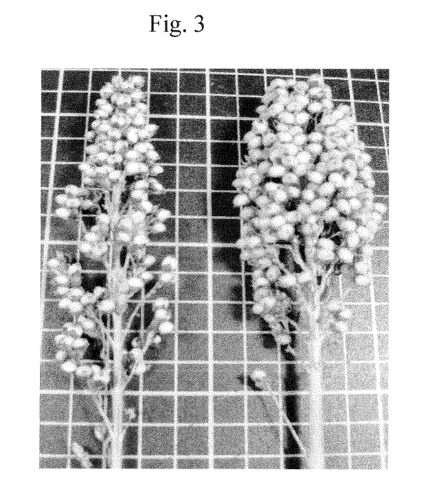

FIG. 4: Variation in seed size in anti-CCoAOMT plants (left) in comparison to control plants (right)

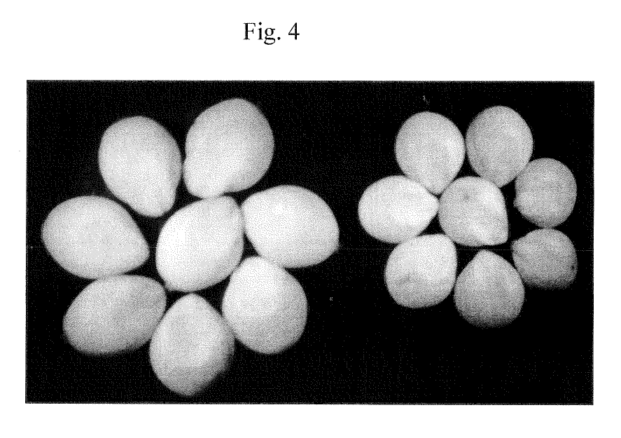

FIG. 5: Double strand mediated anti-CCoAOMT construct that will facilitate the specific in vivo production of RNAi against endogenous CCoAOMT gene.

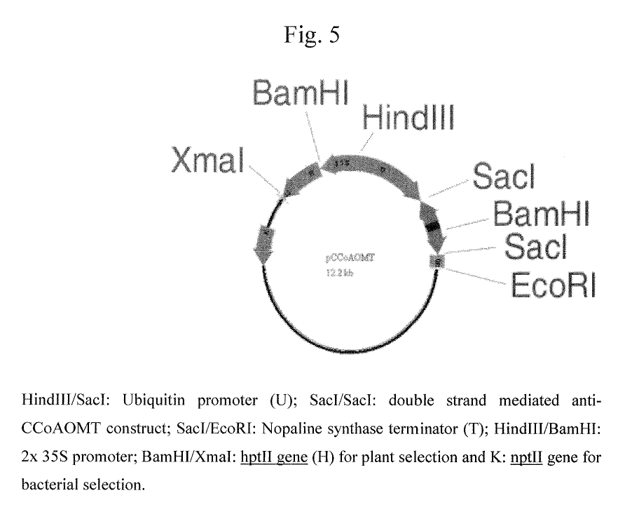

FIG. 6: Selection of plants by PCR amplification of marker genes of transformed lines of COMT and CCoAOMT.

FIG. 7: Southern analysis of T₁ progenies of CCoAOMT along with the respective T₀ transformants and a control plant showing inheritance of the integrated transgenes.

### DETAILED DESCRIPTION OF THE PRESENT INVENTION

Accordingly, the present invention provides a sweet sorghum plant characterized by altered lignin composition compared to a wild plant and this is achieved by down regulating the expression of polynucleotide represented by SEQ ID NO 1 in sweet sorghum.

One of the aspects of the invention is RNAi mediated gene silencing of the endogenous gene represented by SEQ ID NO 1 such that when the exogenous nucleic acid is transcribed, the activity of the endogenous SEQ ID NO 1 is down regulated.

Also provided herewith is a process of producing a modified sweet sorghum plant having altered lignin composition. Said process comprises transfecting a plant cell with double strand RNA (dsRNA) mediated gene silencing cassettes using nucleotide sequence represented in SEQ ID NO 1 that facilitates the in vivo production of RNAi against the endogenous gene and associated with reduced content of G-lignin as compared to the wild plant.

Two transgenic lines were developed, one with anti-COMT and other with anti-SEQ ID NO 1, and the anti-SEQ ID NO 1 transformed plant showed desirable as well as beneficial phenotypic characters like enhanced growth, panicle shape, increased number of seed per panicle, increased size and weight and thereby provide the more biomass as raw material for downstream use and higher starch and free sugar content as compared to anti-COMT transformed plant. The plants transformed with anti-COMT and anti-SEQ ID NO 1 construct hereinafter referred to as “C-plant” and “T-plant” respectively.

The genetic modification of sweet sorghum with SEQ ID NO 1 has a far reaching implication. The intended use of sorghum biomass would be in fermentation process. In fermentation process, it is very important to have biomass with high sugar content and preferably soluble sugar. It is pertinent to mention here that upon morphological and biochemical analysis of the modified sorghum plant, a substantial increase in the biomass and cellulose content was observed.

### Quest for a New Gene in Sweet Sorghum Capable of Altering Lignin Composition:

Isolation of Total RNA from Mature Sorghum Stem by Hot Phenol Method:

1 gm fresh sorghum stem was taken and chopped into small pieces and crushed in mortar with pestle in presence of liquid nitrogen still it becomes powdery. A pinch of poly vinyl pyrrolidone (PVP) and 200 μl β-marcapto ethanol (β-ME) were added with the powder. 5 ml RNA extraction buffer and 5 ml saturated phenol mixed previously and heated in 80° C. water bath was added to the powder, mixed well and allowed to thaw. Thawed sample then transferred in a centrifuge tube and heated in 80° C. water bath for 20 minutes. The tube was kept in room temperature for 5-10 minutes and 5 ml of chloroform was added to it and the tube was vortexed very well. Then the tube was centrifuged at 10,000 rpm for 10 minutes at room temperature, supernatant was taken in a corex tube. 1/10^{th} volume of Na-acetate and 2 volume of chilled ethanol were added to it and allowed to precipitate at −20° C. overnight. Next day the corex tube was centrifuged at 10,000 rpm for 10 minutes at 4° C. Supernatant was discarded and the pellet was air dried. Pellet was dissolved in 1 ml DEPC treated water and distributed into two microfuge tubes. Equal volume of saturated phenol added, shaked gently and centrifuged at 10,000 rpm for 5 minutes and the supernatant was taken. Phenol step was repeated still the supernatant becomes clear. Followed by phenol step equal amount of chloroform was added and centrifuged at 10,000 rpm for 5 minutes, supernatant was taken. 1/10^{th} volume of Na-acetate and 2 volume of chilled ethanol were added to it and allowed to precipitate at −20° C. for 1 hour. It was then centrifuged at 10,000 rpm for 10 minutes and supernatant was discarded, the pellet was washed with 70% ethanol, dried well and finally dissolved in formamide and kept in −70° C. for preservation.

Composition of RNA Extraction Buffer (pH 8):

LiCl—100 mM

TRIS—100 mM

EDTA—10 mM

SDS—1%

Purification of mRNA by Oligo (dT) Cellulose Matrix:

100 mg of oligo (dT) cellulose was suspended in 1 ml elution buffer and kept it at room temperature overnight. Elution buffer was removed by short spin. The column was equilibrated with 1 ml binding buffer. Binding buffer was removed by short spin. 1 mg RNA sample in formamide was precipitated on the previous night and washed with 70% ethanol, dried and dissolved thoroughly in 1 ml binding buffer and heated at 65° C. for 5 minutes, and quickly chilled on ice for 5 minutes. Then the RNA sample was loaded into oligo dT cellulose matrix. Binding was allowed for 30 minutes with gentle shaking. This suspension was centrifuged by short spin. The column was washed 3 times for 30 minutes each with 1 ml wash buffer to remove the binding buffer. Poly (A⁺) mRNA was extracted twice with 200 μl elution buffer, centrifuged at 13,000 rpm for 30 seconds. The eluted pool was readjusted with 0.5M NaCl by adding NaCl accordingly. The whole procedure was repeated twice more from the step of addition of binding buffer to elution. Re-bound, re-washed, re-eluted pooled sample was precipitated with 1/10^{th} volume of Na-acetate and 2.5^{th} volume of ethanol for 1 hour. Pellet was washed with 70% ethanol, dried and re-suspended in DEPC treated water.

Composition of the Buffers:

a) Oligo(dT) Binding Buffer:
TRIS-HCl (pH 7.5)—10 mM
NaCl—500 mM
EDTA—1 mM
SDS—0.5%

b) Oligo(dT) Wash Buffer:
TRIS-HCl (pH 7.5)—10 mM
NaCl—100 mM
EDTA—1 mM

c) Oligo(dT) Elution Buffer:
TRIS-HCl (pH 7.5)—10 mM
EDTA—1 mM
   Synthesis of cDNA from mRNA Isolated from Mature Stem Tissue:

Using reverse transcription, cDNA was prepared from mRNA, which was isolated from sweet sorghum. A primer is annealed to the mRNA providing a free 3′ end that can be used for extension by the enzyme reverse transcriptase. The enzyme engages in the usual 5′ to 3′ elongation, as directed by complementary base pairing with the mRNA template to form a hybrid molecule, consisting of a template RNA strand base paired with the complementary cDNA strand. After degradation of the original mRNA, a DNA polymerase was used to synthesize the complementary DNA strand to convert the single stranded cDNA into a duplex cDNA.

Desired complete cDNA was isolated using PCR (Polymerase Chain reaction) with degenerated primers designed from conserved amino acid sequence of the gene from heterologous plant system followed by 5′ and 3′ RACE (Rapid Amplification of cDNA Ends). cDNA was synthesized with anchored oligo(dT)₁₈ primer and random hexamer primer using Standard RT-PCR Reaction kit by Roche. In a sterile, nuclease free, thin walled PCR tube on ice, template primer mixture was prepared for one 20 μl reaction by adding the components in the order listed below.

Template-Primer Mix:

| Component | Volume | Final conc. |
|---|---|---|
| Poly(A)⁺ mRNA | 1 μl | 10 ng poly(A)⁺ |
| | | mRNA |
| Anchored-oligo(dT)₁₈ primer, 50 | 1 μl | 2.5 μM |
| pmol/μl | | |
| Random hexamer primer, 600 | 2 μl | 60 μM |
| pmol/μl | | |
| PCR grade water | 9 μl | |
| Total volume | 13 μl | |

The template-primer mixture was heated for 10 minutes at 65° C. in a thermal block cycler with a heated lid to denature the secondary structures. Immediately the tube was placed on ice. The remaining components of the RT mix were added in the order listed below.

| | | |
|---|---|---|
| Component | Volume | Final conc. |

| | | | |
|---|---|---|---|
| Transcriptor Reverse | 4 | μl | 1X (8 mM |
| Transcriptase Reaction Buffer (5X) | | | MgCl₂) |
| Protector RNase Inhibitor, 40 U/μl | 0.5 | μl | 20 U |
| Deoxynucleotide Mix, 10 mM | 2 | μl | 1 mM |
| each | | | each |
| Transcriptor Reverse | 0.5 | μl | 10 U |
| Transcriptase, 20 U/μl | | | |
| Final volume | 20 | μl | |

The reagents were mixed carefully and centrifuged briefly and kept at thermal block cycler for 10 minutes at 25° C., followed by 30 minutes at 55° C. Transcriptor Reverse Transcriptase was inactivated by heating it to 85° C. for 5 minutes. The reaction was stopped by placing the tube on ice.

### Design and Synthesis of Primers and PCR Amplification:

Two degenerated 5′ and 3′ primers were designed. A BamHI site in the 5′-primer and an EcoRI site in the 3′-primer were introduced for cloning of the PCR amplified fragment. Translated amino acid sequence analysis of the amplified fragment showed identity with the heterologous CCoAOMT. The sequences of the degenerate primers are:

Forward primer: 5′ gaa ttc gga tcc a(c/t)c a(a/g)g a(a/g)g tng gnc a(c/t)a a 3′ (SEQ ID NO 3)

Reverse primer: 5′ gaa ttc (g/a)tt cca nag ngt (g/a)tt (g/a)tc (g/a)ta 3′ (SEQ ID NO 4)

| | Total | Temperature | Duration |
|---|---|---|---|
| Steps | cycle | (° C.) | (min) |
| Primary | 1 | 94 | 4 |
| denaturation | | | |
| Denaturation | 30 | 94 | 0.4 |
| Annealing | | 58 | 0.4 |
| Extension | | 72 | 1 |
| Final | 1 | 72 | 7 |
| extension | | | |

### Cloning of the Partial SEQ ID NO 1 Fragment:

Both the PCR amplified fragment and vector pUC18 was digested with restriction enzymes BamHI and EcoRI for cloning as it was introduced in the primers. Both the digested vector and amplified fragment were purified by LMP agarose gel. Purified products were subjected to ligation with T4 DNA ligase. A part of the ligation mixture was then transformed in DH10B competent cells and plated on ampicillin (100 m/ml). Transformed colonies were selected. The recombinant plasmid DNA was isolated from the clones and sequenced. The cloned fragment was further amplified with specific primers (SEQ ID NO 5 and 6) and digested with BamHI and SacI and joined in sense-antisense orientation with the help of arbitrary linker having BamHI and BgIII sites. This sense-antisense fragment was then placed in a binary vector under a suitable promoter to generate dsRNA induced cassette for CCoAOMT for Agrobacterium mediated transformation in plant.

The sequences of the specific primers are:

Forward primer:
(SEQ ID NO 5)
5′ gaa ttc gga tcc cat cag gaa gta ggg cac aa 3′
Reverse primer:
(SEQ ID NO 6)
5′ cc gag ctc gtt cca cag tgt gtt gtc ata 3′

### Isolation of Full-Length SEQ ID NO 1:

The full-length SEQ ID NO 1 was isolated through amplification of 5′ and 3′ ends from cDNA of sorghum using 2^{nd} generation 5′/3′ RACE kit in stepwise manner. The fragments were then cloned and characterized. The primers were generated from the identified nucleotide sequence of partial SEQ ID NO 1.

### Cloning of the SEQ ID NO 1 in TA-Vector:

The PCR amplified fragment was purified by LMP agarose gel and cloned in TA vectors. A part of the ligation mixture was then transformed in DH10B competent cells and plated on ampicillin (100 μg/ml). Transformed colonies were selected and characterized by sequencing.

### Characterization of the Clones and Software Based Analysis of the Sequenced Clones:

Selected white clones were initially characterized by isolating plasmid DNA and restriction digestion with BamHI and EcoRI for the presence of the insert. Then three of these clones for each fragments were sequenced by Big Dye Terminator method of sequencing for further confirmation using software based analytical system like NCBI blast analysis and Clustal W alignment.

Nucleotide Sequence as Obtained from Sequencing Result:

The sequence of the gene was found to be 759 bases. The coding DNA sequence of the gene was found to start from base at 1 and end at base 759. The nucleotide sequence hereinafter referred to as SEQ ID NO 1 and is as follows:

(SEQ ID NO 1)
5′-ATGGCCGAAAACGGCGAAGAGCAGCAGGCGAACGGCAACGGCGAGCAGAAGACCCGG
CATCAGGAAGTAGGGCACAAGAGCCTGCTCAAGAGCGACGAGCTCTACCAGTACATCCT
GGACACGAGCGTGTACCCGCGGGAGCCGGAGAGCATGAAGGAGCTCCGCGAGATCACC
GCCAAGCACCCATGGAACCTGATGACGACCTCCGCCGACGAGGGGCAGTTCCTCAACAT
GCTCATCAAGCTCATCGGCGCCAAGAAGACCATGGAGATCCGCGTCTACACCGGCTACTC
CCTCCTTGCTACTGCCATGGCTCTTCCCGATGATGGCAAGATTCTAGCTATGGATATTAAC
CGGGAAAACTACGAGATTGGTCTTCCAGTGATTGAAAAGGCTGGACTGGCCCACAAGAT
CGACTTCCGCGAGGGCCCCGCGCTCCCCGTCCTCGACGACCTCATCGCCGACGAGAAGA
ACCACGGGTCGTTCGACTTCGTCTTCGTGGACGCCGACAAGGACAACTACCTCAACTACC
ACGACCGGCTGCTCAAGCTGGTGAAGCTGGGGGGCCTCATCGGCTATGACAACACACTG
TGGAACGGGAGCGTCGTGCTGCCCGACGACGCCCCGATGCGGAAGTACATTCGCTTCTA
CCGCGATTTCGTCCTCGTCCTGAACAAGGCGCTCGCGGCGGATGATCGCGTCGAGATCTG
CCAGCTCCCCGTCGGTGACGGTGTGACGCTGTGCCGGCGCGTCAAGTGA-3′

### Computer Based Analysis of the Sequence for Further Characterization:

The DNA sequence was then translated to get the amino acid sequence using Jellyfish software. The translated sequence is:

(SEQ ID NO 2)
MAENGEEQQANGNGEQKTRHQEVGHKSLLKSDELYQYILDTSVYPREPESMKELREITAKHP
WNLMTTSADEGQFLNMLIKLIGAKKTMEIRVYTGYSLLATAMALPDDGKILAMDINRENYEI
GLPVIEKAGLAHKIDFREGPALPVLDDLIADEKNHGSFDFVFVDADKDNYLNYHDRLLKLVKLG
GLIGYDNTLWNGSVVLPDDAPMRKYIRFYRDFVLVLNKALAADDRVEICQLPVGDGVTLCRR
VK*

This translated amino acid sequence was then subjected to blast analysis using blastP in NCBI and maximum homology was found with its close relative CCoAOMT of Zea mays. Thus, the sequence was further analyzed for homology with the CCoAOMT isoforms of maize available in the Genbank database using Clustal W software (shown below) and revealed 95% identity with maize CCoAOMT isoform.

| | | |
|---|---|---|
| Maize CCoAOMT1 | ------MATTATEAAPAQEQQANGNGEQKTRHSEVGHKSLLKSDDLYQYILDTSVYPREP | (SEQ ID NO 7) |
| Sorghum CCoAOMT | ------------MAENGEEQQANGNGEQKTRHQEVGHKSLLKSDELYQYILDTSVYPREP | (SEQ ID NO 8) |
| Maize CoAOMT2 | MATTATEATKTTAPAQEQQANGNGNGEQKTRHSEVGHKSLLKSDDLYQYILDTSVYPREP | (SEQ ID NO 9) |
| Rice CCoAOMT | ----MAEAASAAAAATTEQANGSSGGEQKTRHSEVGHKSLLKSDDLYQYILETSVYPREH | (SEQ ID NO 10) |
| | . :::....*******.***********:******:******* | |
| Maize CCoAOMT1 | ESMKELREVTAKHPWNLMTTSADEGQFLNMLIKLIGAKKTMEIGVYTGYSLLATALALPE | (SEQ ID NO 11) |
| Sorghum CCoAOMT | ESMKELREITAKHPWNLMTTSADEGQFLNMLIKLIGAKKTMEIRVYTGYSLLATAMALPD | (SEQ ID NO 12) |
| Maize CCoAOMT2 | ESMKELREITAKHPWNLMTTSADEGQFLNMLIKLIGAKKTMEIGVYTGYSLLATALALPE | (SEQ ID NO 13) |
| Rice CCoAOMT | ECMKELREVTANHPWNLMTTSADEGQFLNLLLKLIGAKKTMEIGVYTGYSLLATALAIPD | (SEQ ID NO 14) |
| | *.******:**:*****************:*:*********** ***********:*:*: | |
| Maize CCoAOMT1 | DGTILAMDINRENYELGLPCIEKAGVAHKIDFREGPALPVLDDLIAEEKNHGSFDFVFVD | (SEQ ID NO 15) |
| Sorghum CCoAOMT | DGKILAMDINRENYEIGLPVIEKAGLAHKIDFREGPALPVLDDLIADEKNHGSFDFVFVD | (SEQ ID NO 16) |
| Maize CCoAOMT2 | DGTILAMDINRENYELGLPCINKAGVGHKIDFREGPALPVLDDLVADKEQHGSFDFAFVD | (SEQ ID NO 17) |
| Rice CCoAOMT | DGTILAMDINRENYELGLPSIEKAGVAHKIDFREGPALPVLDQLVEEEGNHGSFDFVFVD | (SEQ ID NO 18) |
| | **.************:*** *:***:.***************:*: :: :******.*** | |
| Maize CCoAOMT1 | ADKDNYLNYHERLLKLVKLGGLIGYDNTLWNGSVVLPDDAPMRKYIRFYRDFVLVLNKAL | (SEQ ID NO 19) |
| Sorghum CCoAOMT | ADKDNYLNYHDRLLKLVKLGGLIGYDNTLWNGSVVLPDDAPMRKYIRFYRDFVLVLNKAL | (SEQ ID NO 20) |
| Maize CCoAOMT2 | ADKDNYLNYHERLLKLVRPGGLIGYDNTLWNGSVVLPDDAPMRKYIRFYRDFVLALNSAL | (SEQ ID NO 21) |
| Rice CCoAOMT | ADKDNYLNYHERLMKLVKVGGLVGYDNTLWNGSVVLPADAPMRKYIRYYRDFVLELNKAL | (SEQ ID NO 22) |
| | **********:**:***: ***:************** *********:****** **.** | |
| Maize CCoAOMT1 | AADDRVEICQLPVGDGVTLCRRVK | (SEQ ID NO 23) |
| Sorghum CCoAOMT | AADDRVEICQLPVGDGVTLCRRVK | (SEQ ID NO 24) |
| Maize CCoAOMT2 | AADDRVEICQLPVGDGVTLCRRVK | (SEQ ID NO 25) |
| Rice CCoAOMT | AADHRVEICQLPVGDGITLCRRVK | (SEQ ID NO 26) |
| | ***.************:******* | |

### Assessing the Role of SEQ ID NO 1 in Sweet Sorghum:

The sequence was then subjected to NCBI blast for identification of its resemblance with the sequence available in the database and found to be highly identical (97% identity) with a hypothetical protein of sorghum. Though this hypothetical protein has been considered to similar to CCoAOMT gene, but its functionality has not yet been tested. Thus, we have taken an approach to establish the effect of isolated gene, as the nucleotide sequence of a gene is more important rather than its functionality in antisense strategy, on down-regulation of CCoAOMT in-planta.

Preparations of Construct comprising SEQ ID NO 1:

Promoter:

Transcription of DNA into mRNA is regulated by a region of DNA referred to as the promoter. The promoter region contains sequence of bases that signals RNA polymerase to associate with the DNA, and to initiate the transcription of mRNA using one of the DNA strands as a template to make a corresponding complementary strand of RNA. Since the 5′ region of the RNA strand is complementary to the 3′ region, it will generate a double-stranded RNA, which subsequently degraded using machinery responsible for the production short interfering RNA (RNAi). Promoter sequence include the TATA box consensus sequence (TATAAT (SEQ ID NO 27)), which is usually 20-30 base pair (bp) upstream (by convention −30 to −20 bp relative to the transcription start site) of the transcription start site. The TATA box is the only upstream promoter element that has a relatively fixed location with respect to the start point. The CAAT box consensus sequence is centered at −75, but function at distances that vary considerably from the start point and in either orientation. Another common promoter element is the GC box at −90 which contains consensus sequence GGGCGG (SEQ ID NO 28). It may occur in multiple copies and in either orientation. Other sequence conferring maximum efficiency may also be found in the promoter region. In promoter and structural gene combinations, the promoter is preferably in positioned about the same distance from the heterologous transcription start site as it is from the transcription start site in its natural setting. The promoter can be either constitutive or inducible.

The maize ubiquitin promoter used in the present investigation has been shown to be highly active and constitutively expressed in most tissues. It contains the first intron of the maize ubiquitin gene for selective expression in plants. The promoter was cloned in the vector at HindIII/BamHI site under which the dsRNA inducing fragment of SEQ ID NO 1 was placed. A selectable marker gene, hygromycin phosphotransferase, under 2×35S promoter was included to allow selection of plant cells bearing the desired construct.

Generation of dsRNA construct was made by joining the suitable region of the desired cDNA fragment in sense and antisense orientation through linker. This construct is introduced into an expression vector for transformation of sorghum plants to find out what role it plays in the metabolic pathway. The vector preferably contains a prokaryotic origin of replication having a broad host range. A selectable marker should also be included to allow selection of bacterial cells bearing the desired construct. Suitable prokaryotic selectable markers include resistance to antibiotics such as kanamycin.

Other DNA sequences encoding additional functions may also be present in the vector, as is known in the art. For instance, in the case of Agrobacterium transformations, T-DNA sequences will also be included for subsequent transfer to plant chromosomes.

For expression in plant, a binary vector was used in which gene of interest can be introduced. The recombinant expression cassettes will contain in addition to desired sequences, a plant promoter region, a transcription initiation site and a transcription terminator sequence. Unique restriction enzyme site at the 5′ and 3′ ends of the cassettes are typically included to allow for easy insertion into a pre-existing vector. Sequences controlling eukaryotic gene expression are well known in the art.

Generation of Transgenic Plants:

Two transgenic lines were developed. The plants transformed with anti-COMT construct hereinafter referred to as “C-plant” and plant having anti-SEQ ID NO 1 referred to as “T-plant”. T-plant showed desirable as well as beneficial phenotypic characters like enhanced growth, panicle shape, increased number of seed per panicle, increased size and weight, and thereby provides more biomass as raw material for downstream use.

Transformation of Sweet Sorghum (Sorghum bicolor):

Seeds surface sterilized with Tween 20 (5 min) and 0.2% mercuric chloride (7 min) were washed with sterile distilled water. Then seeds were incubated on sterile filter paper soaked with sterile distilled water in Petri plates. After 3 days incubation in dark the shoot tips generated were excised and infected with infection medium having Agrobacterium suspension in it for 20 min. The explants were inoculated on co-cultivation medium and kept in dark for 3 days at 25° C. The explants were occasionally washed with cefotaxime and distilled water to prevent bacterial contamination and transferred on MS with 2 mg/l 2,4-D, 30 gm/l sucrose and 250 mg/l cefotaxime and kept in dark for 12 days for callus formation. Callus portion at the cut ends of shoot tips were excised and transferred to regeneration medium with hygromycin selection (MS with 30 g/l sucrose, 2 mg/l BAP and 2 mg/l hygromycin) and kept them in 2:1 light/dark periodic condition at 28° C. After 2 weeks, green calli were transferred on the same medium containing higher concentration of selection marker (4 mg/l hygromycin). Shoots obtained were transferred on the same medium with higher concentration of hygromycin (5 mg/l) for 2 months with periodic sub-culturing every 2 weeks. Elongated shoots were allowed to rooting medium for root generation. Full grown plantlets were finally selected on ½ MS liquid medium with 6 mg/l hygromycin. Plantlets generated from a single callus were described as single line.

### The Agrobacterium-Mediated Transformation:

The Agrobacterium-mediated transformation process of the invention can be broken into several steps. The basic steps include an infection step; a co-cultivation step; an optional resting step; a selection step; and a regeneration step.

In the infection step, the cells to be transformed are isolated and exposed to Agrobacterium. If the target cells are immature embryos, the embryos are isolated and the cells contacted with a suspension of Agrobacterium. As noted above, the Agrobacterium has been modified to contain a gene or nucleic acid of interest. The nucleic acid is inserted into the T-DNA region of the vector. General molecular techniques used in the invention are provided, for example, by Sambrook et al. (eds.) Molecular Cloning: A Laboratory Manual, 1989, Cold Spring Harbor Laboratory Press, Cold Spring Harbor, N.Y.

The concentration of Agrobacterium used in the infection step and co-cultivation step can affect the transformation frequency. Likewise, very high concentrations of Agrobacterium may damage the tissue to be transformed, such as the immature embryos, and result in a reduced callus response. Thus, the concentration of Agrobacterium useful in the methods of the invention may vary depending on the Agrobacterium strain utilized, the tissue being transformed, the sorghum genotype being transformed, and the like. To optimize the transformation protocol for a particular sorghum line or tissue, the tissue to be transformed, (immature embryos, for example), can be incubated with various concentrations of Agrobacterium. Likewise, the level of marker gene expression and the transformation efficiency can be assessed for various Agrobacterium concentrations. While the concentration of Agrobacterium may vary, generally optical density 0.7 to 1.0 at 600 nm was used in the present invention.

The tissue to be transformed is generally added to the Agrobacterium suspension in a liquid contact phase containing a concentration of Agrobacterium to optimize transformation efficiencies. The contact phase facilitates maximum contact of the cells/tissue to be transformed with the suspension of Agrobacterium. The cells are contacted with the suspension of Agrobacterium for a period of at least about three 3 minutes to about 15 minutes, preferably about 4 minutes to about 10 minutes, more preferably about 5 minutes to about 8 minutes

The liquid contact phase of the infection step takes place in a liquid solution MS media along with 68.5 g/l sucrose, 36 g/l glucose, 100 μM acetosyringone and the pH adjusted to 5.2. The other media used in this invention are: Co-cultivation media (MS with 20 g/l sucrose, 10 g/l glucose, 2 mg/l 2,4-D, 100 μM acetosyringone and 8.5 g/l agar, pH 5.8), Bacterial culture media (YEP Media—Yeast extract—10 g/l, Peptone—10 g/l and Sodium Chloride—5 g/l), Infection Media (MS with 68.5 g/l sucrose, 36 g/l glucose and 100 μM acetosyringone, pH 5.2), Regeneration Media (MS with 30 g/l sucrose, 2 mg/l BAP and 8.5 g/l agar) and Rooting Media (½ MS with 20 g/l sucrose, 0.5 mg/l IAA and 0.5 mg/l NAA)

Concentration of Agrobacterium During Infection:

O.D. of Agrobacterium—Between 0.7-1.0

Following the co-cultivation step, or following the resting step, where it is used, the transformed cells are exposed to selective pressure to select for those cells that have received and are expressing polypeptide from the heterologous nucleic acid introduced by Agrobacterium. Where the cells are embryos, the embryos are transferred to plates with solid medium that includes both an antibiotic to inhibit growth of the Agrobacterium and a selection agent. The agent used to select for transformants will select for preferential growth of explants containing at least one selectable marker insert positioned within the super binary vector and delivered by the Agrobacterium.

Generally, any of the media known in the art suitable for the culture of sorghum can be used in the selection step, such as media containing N6 salts or MS salts supplemented with 30 g/l sucrose, 2 mg/l 2,4-D and kept in dark for 15 days. During selection, the embryos are cultured until callus formation is observed. Typically, calli grown on selection medium are allowed to grow to a size of about 1.5 to about 2 cm. diameter.

After the calli have reached the appropriate size, the calli are cultured on regeneration medium in the dark for several weeks, generally about 1 to 3 weeks to allow the somatic embryos to mature. Preferred regeneration media include media containing MS media supplemented with 30 g/l sucrose, 2 mg/l BAP and 8.5 g/l agar. The calli are then cultured on rooting medium in a light/dark cycle until shoots and roots develop

Small plantlets are then transferred to tubes containing rooting medium and allowed to grow and develop more roots for approximately another week. The plants are then transplanted to soil mixture in pots in the greenhouse.

Screening of the dsRNA Induced Putative Transformed Lines:

The putative T₀ transformants for both the dsRNA inducing transgenes recovered through hygromycin selection were further screened by PCR analysis to confirm the presence of hygromycin resistant gene. Thus, a PCR analysis was made using the genomic DNA isolated from the transformed individuals along with non-transformed control plant and a set of gene specific primers (5′ primer: atg aaa aag cct gaa ctc acc gcg acg tct and 3′ primer: gca tca get cat cga gag cct gcg cga cgg). An amplification of about 547 bp product was observed in all the transformants indicated the presence of hygromycin resistance gene. The amplification was found to be absent in non-transformed Sorghum plant. Thus, it could be expected that dsRNA inducing COMT and CCoAOMT gene cassettes might have inserted in the genome of the putative T₀ transformants as it was kept linked with the hygromycin resistance gene (hptII).

### Southern Analysis of Transgenic Plants:

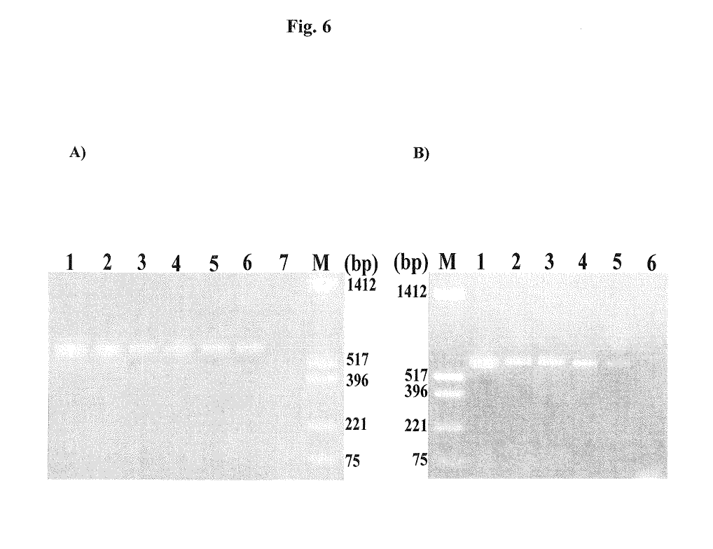

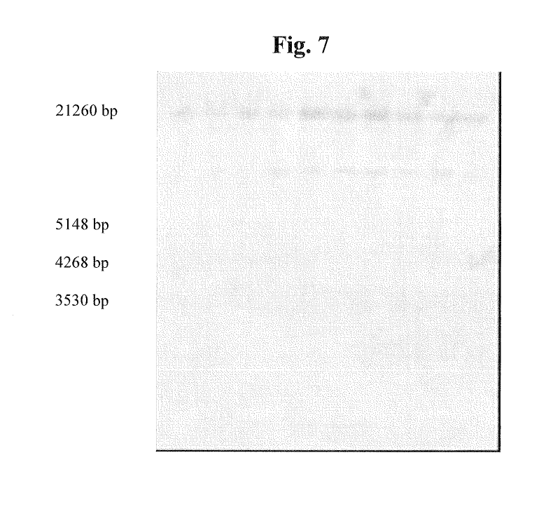

Southern analysis of transgenic plants was done to check the integration pattern as well as copy number of the integrated gene construct. For this, DNA was isolated from control, T0 and T1 plants. 10 mg of DNA of each of the plants was digested with HindIII and run in a 1% agarose gel. The gel was then transferred onto Nylon membrane (GE healthcare), cross-linked and finally hybridized with COMT and CCoAOMT gene probes respectively (FIGS. 6 and 7).

T1 progenies were produced from seeds of self-pollination of T0 plants. Seeds were screened by allowing them to grow in the basal medium containing hygromycin. Eight seeds were found to germinate under hygromycin. The resistant seedlings were then allowed to grow to a mature stage in containment. The plants were analysed for the presence of hygromycin gene by PCR using two gene-specific primers. Finally, Southern analysis of the T1 transgenic plants was done to check the integration pattern as well as copy number of the integrated gene construct and single integration was observed in the anti-SEQ ID NO 1 transgenic plants except in T1-1. Moreover, the integration pattern was found similar in all cases. The enzyme activity in T1 plants was found to be almost same in comparison to the T0 plant.

### Analysis of Endogenous Enzyme Activities in Transformed Lines:

Enzyme activities were analyzed in the tissue of transformed sweet sorghum to assess the alteration of lignin composition. The results have been tabulated in Table 1. Table 2 clearly indicates that down regulation of SEQ ID NO 1 resulted into reduction in lignin content. The result showed that lignin content in stem tissue was reduced to about 14% and 27% in C-plants and T-plants respectively. Table 3 discloses S/G ratio in the transformed plants and control as well. The results showed that S/G ratio was increased in both the plants. The change in S/G ratio was more prominent in T-plant. So, it could be expected that this increase in S/G ratio facilitate the extractability of the cellulosic materials from both the transgenic plant, particularly from T-plant.

TABLE 1
Enzyme activity in stem tissue of C and T plants:

| Lines | COMT activity | CCoAOMT |
|---|---|---|
| activity | (pmol/sec/mg of total protein) | (pmol/sec/mg of total protein) |
| Control | 4.8 | 14.3 |
| C-plants | 2.3 | 15.3 |
| T-plants | 4.6 | 4.9 |

### Alteration in Growth Parameters of Transgenic Lines:

The transgenic lines showed alteration of growth when compared with control. The increment in the plant height was found to be co-related with the increase of internodal length as well as number of internodes. Apart from the increased vegetative growth, an alteration was also observed in seed sizes in transgenic plants compared to control plant. This increase in seed sizes was verified by mean seed weight. The increment was found to be ^{˜}45% over the control seeds.

### Biochemical Analysis of Transformed Sweet Sorghum Plants:

Analysis of lignin content and composition in transgenic plants by chemical method:

Lignin composition with respect to G and S type was determined by derivatization followed by reductive cleavage (DFRC). The monomeric lignin degradation products were identified by GC-FID analysis using acetyl derivative of coniferyl and sinapyl alcohol as standard. The reduction of total lignin content compared to control was ^{˜}28% in case of T-plant. But the reduction was to a lesser degree (_{˜}14%) in C-plant.

TABLE 2
Determination of lignin concentration of transformed Sorghum
stem using the Acetyl Bromide Spectrophotometric method:

| | Acetyl Bromide |
|---|---|
| | lignin content |
| Stem tissue | (gm/kg dry cell wall) |
| Control plant | 76.14 |
| C- plant | 65.65 |
| T- plant | 54.84 |

TABLE 3
Lignin composition of stem tissue of C and T plants.

| Sorghum | | | |
|---|---|---|---|
| plant | S type (%) | G type (%) | S/G ratio |
| Control | 22.29 | 77.71 | 0.29 |
| plant | | | |
| C-plants | 39.99 | 60.01 | 0.66 |
| T-plants | 63.51 | 36.49 | 1.74 |

### Estimation of Cellulose:

A segment from stem (20 mg by fresh weight) of control and transgenic sorghum plants were collected, frozen in liquid nitrogen and crushed to a fine powder. The powder was treated with 3 ml of acetic/nitric reagent (10:1) in boiling water bath for 30 mins, cool and then centrifuged for 15-20 mins. The supernatant was stored to estimate the soluble sugar content. The pellet was washed, treated with 67% sulphuric acid and allowed to stand for 1 hr. The treated sample was then diluted 15 times. 5 ml of anthrone reagent was added to 1 ml of both the treated and untreated sample, mix well, kept in boiling water bath for 10 mins, cooled rapidly and measured O.D. at 630 nm. The results were expressed as a mean of three samples in each case.

The carbohydrate content of both seed and stem tissue was estimated following the protocols described in Methods. It has been observed that the cellulose content was increased by 48% in C-plants and by 36% in T-plants (Table 4). It is important to note that in spite of higher cellulose content in C-plants (Table 4), the soluble sugar was found to increase more significantly in T-plants (Table 5). Higher content of soluble sugar would help in fermentation process.

TABLE 4
Determination of cellulose content in sorghum stem tissue

| | Cellulose content |
|---|---|
| Samples | (mg/g of FW) |
| Control | 86 |
| Anti-COMT line | 127 |
| Anti-CCoAOMT-like line | 117 |

TABLE 5
Determination of soluble sugar content in sorghum stem tissue

| | Soluble sugar content |
|---|---|
| Samples | (mg/g of FW) |
| Control | 10.3 |
| Anti-COMT line | 12.1 |
| Anti-CCoAOMT-like line | 29.5 |

### Estimation of Starch in Seeds:

Seeds were homogenized in hot 80% ethanol to remove sugar and centrifuged. The pellet was washed repeatedly with 80% ethanol and then dried. The pellet was treated with 5 ml of water and 6.5 ml of 52% perchloric acid for 20 mins. The extract was centrifuged and the supernatant was collected. The supernatant was diluted 250 times. 5 ml anthrone reagent was added to 1 ml of the sample, kept at boiling water bath for eight mins and measured at O.D. 630 nm. The results were expressed as a mean of three samples in each case.

The starch content of both seed and stem tissue was estimated following the protocols described in Methods. It has also been observed that starch content in seeds was increased by around 20 and 30% in the C-plants and T-plants respectively (Table 3). It could be suggested that accumulation of starch, apart from other components, may be responsible for increase of seed weight.

TABLE 6
Determination starch content in seed

| Samples | Average Seed weight | Average Starch |
|---|---|---|
| content | (mg) | (mg/seed) |
| Control | 29.4 | 16.6 |
| Anti-COMT line | 38.3 | 20.2 |
| Anti-CCoAOMT-like line | 42.3 | 22.1 |

Throughout this application, various patents and publications have been cited. The disclosures of these patents and publications in their entireties are hereby incorporated by reference into this application, in order to more fully describe the state of the art to which this invention pertains.

While the present invention has been described for what are presently considered the preferred embodiments, the invention is not so limited. To the contrary, the invention is intended to cover various modifications and equivalent arrangements included within the spirit and scope of the detailed description provided above.

We claim:

1. An isolated polynucleotide sequence obtained from Sorghum bicolor encoding Caffeoyl-CoA-O-methyltransferase (CCoAOMT) represented by SEQ ID NO 1.

2. An isolated polynucleotide sequence as claimed in claim 1, wherein the DNA is cDNA.

3. A double strand mediated gene silencing cassette comprising a nucleic acid represented in SEQ ID NO 1, said nucleic acid is operably associated with a promoter, wherein the nucleic acid is in the sense-antisense orientation and facilitate to generate RNAi in vivo to at least a portion of a SEQ ID NO 1.

4. Forward and reverse primers represented by SEQUENCE ID NO 5 and 6.

5. A vector comprising a construct of claim 3.

6. A vector as claimed in claim 5, wherein the vector used is a binary vector.

7. A transgenic plant comprising a cassette of claim 3.


SEQUENCE LISTING (31 sequences)

SEQ ID NO: 1 - DNA, 759 bp, Artificial Sequence
  Description of Artificial Sequence Synthetic cDNA of CaffeoylCoA-O-methyltransferae CCoAOMT isolated from sweet sorghum (Sorghum bicolor)
atggccgaaaacggcgaagagcagcaggcgaacggcaacggcgagcagaagacccggcatcaggaagtagggcacaagagcctgctcaagagcgacgagctctaccagtacatcctggacacgagcgtgtacccgcgggagccggagagcatgaaggagctccgcgagatcaccgccaagcacccatggaacctgatgacgacctccgccgacgaggggcagttcctcaacatgctcatcaagctcatcggcgccaagaagaccatggagatccgcgtctacaccggctactccctccttgctactgccatggctcttcccgatgatggcaagattctagctatggatattaaccgggaaaactacgagattggtcttccagtgattgaaaaggctggactggcccacaagatcgacttccgcgagggccccgcgctccccgtcctcgacgacctcatcgccgacgagaagaaccacgggtcgttcgacttcgtcttcgtggacgccgacaaggacaactacctcaactaccacgaccggctgctcaagctggtgaagctggggggcctcatcggctatgacaacacactgtggaacgggagcgtcgtgctgcccgacgacgccccgatgcggaagtacattcgcttctaccgcgatttcgtcctcgtcctgaacaaggcgctcgcggcggatgatcgcgtcgagatctgccagctccccgtcggtgacggtgtgacgctgtgccggcgcgtcaagtga

SEQ ID NO: 2 - protein, 252 aa, Artificial Sequence
  Description of Artificial Sequence Synthetic amino acid sequence translated from SEQ ID NO 1 using Jellyfish software
MAENGEEQQANGNGEQKTRHQEVGHKSLLKSDELYQYILDTSVYPREPESMKELREITAKHPWNLMTTSADEGQFLNMLIKLIGAKKTMEIRVYTGYSLLATAMALPDDGKILAMDINRENYEIGLPVIEKAGLAHKIDFREGPALPVLDDLIADEKNHGSFDFVFVDADKDNYLNYHDRLLKLVKLGGLIGYDNTLWNGSVVLPDDAPMRKYIRFYRDFVLVLNKALAADDRVEICQLPVGDGVTLCRRVK

SEQ ID NO: 3 - DNA, 31 bp, Artificial Sequence
  Description of Artificial Sequence Synthetic forward degenerate primer for amplifying CCoAOMT
gaattcggatccaycargargtnggncayaa

SEQ ID NO: 4 - DNA, 27 bp, Artificial Sequence
  Description of Artificial Sequence Synthetic reverse degenerate primer to amplify CCoAOMT
gaattcrttccanagngtrttrtcrta

SEQ ID NO: 5 - DNA, 32 bp, Artificial Sequence
  Description of Artificial Sequence Synthetic forward primer to amplify CCoAOMT
gaattcggatcccatcaggaagtagggcacaa

SEQ ID NO: 6 - DNA, 29 bp, Artificial Sequence
  Description of Artificial Sequence Synthetic reverse primer to amplify CCoAOMT
ccgagctcgttccacagtgtgttgtcata

SEQ ID NO: 7 - protein, 54 aa, Zea mays
MATTATEAAPAQEQQANGNGEQKTRHSEVGHKSLLKSDDLYQYILDTSVYPREP

SEQ ID NO: 8 - protein, 48 aa, Sorghum bicolor
MAENGEEQQANGNGEQKTRHQEVGHKSLLKSDELYQYILDTSVYPREP

SEQ ID NO: 9 - protein, 60 aa, Zea mays
MATTATEATKTTAPAQEQQANGNGNGEQKTRHSEVGHKSLLKSDDLYQYILDTSVYPREP

SEQ ID NO: 10 - protein, 56 aa, Oryza sativa
MAEAASAAAAATTEQANGSSGGEQKTRHSEVGHKSLLKSDDLYQYILETSVYPREH

SEQ ID NO: 11 - protein, 60 aa, Zea mays
ESMKELREVTAKHPWNLMTTSADEGQFLNMLIKLIGAKKTMEIGVYTGYSLLATALALPE

SEQ ID NO: 12 - protein, 60 aa, Sorghum bicolor
ESMKELREITAKHPWNLMTTSADEGQFLNMLIKLIGAKKTMEIRVYTGYSLLATAMALPD

SEQ ID NO: 13 - protein, 60 aa, Zea mays
ESMKELREITAKHPWNLMTTSADEGQFLNMLIKLIGAKKTMEIGVYTGYSLLATALALPE

SEQ ID NO: 14 - protein, 60 aa, Oryza sativa
ECMKELREVTANHPWNLMTTSADEGQFLNLLLKLIGAKKTMEIGVYTGYSLLATALAIPD

SEQ ID NO: 15 - protein, 60 aa, Zea mays
DGTILAMDINRENYELGLPCIEKAGVAHKIDFREGPALPVLDDLIAEEKNHGSFDFVFVD

SEQ ID NO: 16 - protein, 60 aa, Sorghum bicolor
DGKILAMDINRENYEIGLPVIEKAGLAHKIDFREGPALPVLDDLIADEKNHGSFDFVFVD

SEQ ID NO: 17 - protein, 60 aa, Zea mays
DGTILAMDINRENYELGLPCINKAGVGHKIDFREGPALPVLDDLVADKEQHGSFDFAFVD

SEQ ID NO: 18 - protein, 60 aa, Oryza sativa
DGTILAMDINRENYELGLPSIEKAGVAHKIDFREGPALPVLDQLVEEEGNHGSFDFVFVD

SEQ ID NO: 19 - protein, 60 aa, Zea mays
ADKDNYLNYHERLLKLVKLGGLIGYDNTLWNGSVVLPDDAPMRKYIRFYRDFVLVLNKAL

SEQ ID NO: 20 - protein, 60 aa, Sorghum bicolor
ADKDNYLNYHDRLLKLVKLGGLIGYDNTLWNGSVVLPDDAPMRKYIRFYRDFVLVLNKAL

SEQ ID NO: 21 - protein, 60 aa, Zea mays
ADKDNYLNYHERLLKLVRPGGLIGYDNTLWNGSVVLPDDAPMRKYIRFYRDFVLALNSAL

SEQ ID NO: 22 - protein, 60 aa, Oryza sativa
ADKDNYLNYHERLMKLVKVGGLVGYDNTLWNGSVVLPADAPMRKYIRYYRDFVLELNKAL

SEQ ID NO: 23 - protein, 24 aa, Zea mays
AADDRVEICQLPVGDGVTLCRRVK

SEQ ID NO: 24 - protein, 24 aa, Sorghum bicolor
AADDRVEICQLPVGDGVTLCRRVK

SEQ ID NO: 25 - protein, 24 aa, Zea mays
AADDRVEICQLPVGDGVTLCRRVK

SEQ ID NO: 26 - protein, 24 aa, Oryza sativa
AADHRVEICQLPVGDGITLCRRVK

SEQ ID NO: 27 - DNA, 6 bp, Artificial Sequence
  Description of Artificial Sequence Synthetic oligonucleotide
tataat

SEQ ID NO: 28 - DNA, 6 bp, Artificial Sequence
  Description of Artificial Sequence Synthetic oligonucleotide
gggcgg

SEQ ID NO: 29 - DNA, 18 bp, Artificial Sequence
  Description of Artificial Sequence Synthetic primer
tttttttttttttttttt

SEQ ID NO: 30 - DNA, 30 bp, Artificial Sequence
  Description of Artificial Sequence Synthetic primer
atgaaaaagcctgaactcaccgcgacgtct

SEQ ID NO: 31 - DNA, 30 bp, Artificial Sequence
  Description of Artificial Sequence Synthetic primer
gcatcagctcatcgagagcctgcgcgacgg